(19) United States
(12) Patent Application Publication
(10) Pub. No.: US 2013/0184466 A1
(43) Pub. Date: Jul. 18, 2013

(54) PROCESS FOR PREPARING THE CRYSTALLINE FORM II OF FEBUXOSTAT

(71) Applicants: Josep Salaet Ferré, Barcelona (ES); Francisco Marquillas Olondriz, Barcelona (ES)

(72) Inventors: Josep Salaet Ferré, Barcelona (ES); Francisco Marquillas Olondriz, Barcelona (ES)

(73) Assignee: INTERQUIM, S.A., SANT CUGAT DEL VALLÉS (ES)

(21) Appl. No.: 13/809,839

(22) PCT Filed: Jul. 13, 2011
(86) PCT No.: PCT/EP11/61906

§ 371 Date: Mar. 28, 2013

(30) Foreign Application Priority Data
Jul. 13, 2010 (ES) P201031061

(51) Int. Cl. C07D 277/56

(52) U.S. Cl. CPC C07D 277/56
USPC 548/201

(57) ABSTRACT

The present invention relates to a novel process for preparing the crystalline form II of febuxostat by crystallization of a solvent selected from ethyl acetate, methyl acetate or ethyl formiate.

The present invention relates to a process for preparing the crystalline form II of febuxostat (2-[3-cyano-4-(2-i-butoxy)phenyl]-4-methyl-5-thiazole-carboxylic acid). Febuxostat is an inhibitor of xanthine oxidase that is indicated in the treatment of hyperuricemia. Its structural formula is as follows:

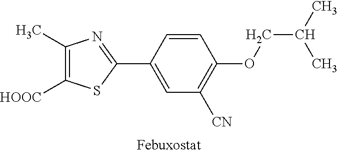 [SMILES: Cc1nc(-c2ccc(OCC(C)C)c(C#N)c2)sc1C(=O)O]

### BACKGROUND ART

CN101139325A relates to two crystalline forms of febuxostat by using ethanol, ethyl acetate or acetone. Among them form II is described, but operational conditions leading to form II are not specifically described. Similarly, CN100546985C and CN101412700A describe the preparation of form II by crystallization from ethyl acetate, but specific operational conditions are not sufficiently described either.

According to different assays performed by the authors of the present invention, it was observed that the preparation of form II under the general conditions disclosed in the above patents does not lead to said polymorph with the sufficient purity degree as to be used in pharmaceutical preparations or reproducibility of the process involved is not sufficiently guaranteed either.

Thus, there is a need to develop a reproducible process for preparing the crystalline form II of febuxostat that is capable of providing a good yield and high purity.

### SUMMARY OF THE INVENTION

The invention provides a reproducible industrial process for preparation of the crystalline form II of febuxostat, which provides a high-yield and highly pure polymorph.

### DETAILED DESCRIPTION OF THE INVENTION

The object of the present invention is to provide a process for preparing the crystalline form II of febuxostat, comprising the following steps:

a) Dissolving febuxostat in a solvent selected from the group consisting of ethyl acetate, methyl acetate and ethyl formiate in a proportion from 10 to 60 ml of solvent per gram of solute, at a temperature between 50° C. and boiling temperature of the solution;

b) Forming the crystals by cooling the solution from step a) at a temperature between 20° C. and 45° C., optionally over a period of 0.5-2 hours under stirring;

c) Cooling the suspension from step b) at a temperature between 0° C. and 30° C. over a period of 0.5-3 hours; and

d) Isolating the crystalline form II of febuxostat by filtration and drying.

In a preferred embodiment, in step a), the proportion of solvent per gram of solute is from 15 to 50 ml.

In another preferred embodiment, in step b), the temperature ranges from 33° C. to 37° C.

In another preferred embodiment, in step b), the period is 1 hour.

In another preferred embodiment, and with the aim of increasing yield of step b), solvent removal (40-80%) between step b) and step c) was performed by distillation under reduced pressure at a temperature from 30° to 40° C.

In a preferred embodiment, the temperature is from 33° C. to 37° C.

### BRIEF DESCRIPTION OF THE DRAWINGS

FIG. 1 shows the X-ray powder diffraction pattern of crystalline form II of febuxostat produced by the process of the present invention. The ordinate shows the intensity value expressed on a linear counting scale and the abscissa shows the diffraction angle (2θ°).

FIG. 2 shows the IR spectrum recorded on KBr tablet of crystalline form II of febuxostat produced by the process of the present invention.

### EXAMPLES

### Example 1

### Preparation of Form II of 2-[3-cyano-4-(2-i-butoxy)phenyl]-4-methyl-5-thiazole-carboxylic acid (Febuxostat) in ethyl acetate

To 10.0 g of 2-[3-cyano-4-(2-i-butoxy)phenyl]-4-methyl-5-thiazole-carboxylic acid, 300 ml of ethyl acetate were added. The mixture was heated at 60° C. until complete dissolution. The solution was cooled to 35° C., and the presence of a precipitate was observed during cooling. The mixture was stirred at 35° C. for 1 hour and 200 ml of solvent were distilled at 35° C. The sample was cooled to 25° C. and kept at this temperature for 1 hour, and then cooled to 0-5° C. and kept at this temperature for 1 hour. The product was filtered and dried under vacuum at 65° C. 9.6 g of febuxostat as pure form II were obtained.

### Example 2

### Preparation of form II of 2-[3-cyano-4-(2-i-butoxy)phenyl]-4-methyl-5-thiazole-carboxylic acid (Febuxostat) in methyl acetate

To 10.0 g of 2-[3-cyano-4-(2-i-butoxy)phenyl]-4-methyl-5-thiazole-carboxylic acid, 250 ml of methyl acetate were added. The mixture was heated under reflux until complete dissolution. The solution was cooled to 35° C., and the presence of a precipitate was observed during cooling. The mixture was stirred at 35° C. for 1 hour and 150 ml of solvent were distilled at 35° C. The sample was cooled to 25° C. and kept at this temperature for 1 hour, and then cooled to 0-5° C. and kept at this temperature for 1 hour. The product was filtered and dried under vacuum at 65° C. 9.4 g of febuxostat as pure form II were obtained.

### Example 3

### Preparation of Form II of 2-[3-cyano-4-(2-i-butoxy)phenyl]-4-methyl-5-thiazole-carboxylic acid (Febuxostat) in ethyl formiate

To 3.0 g of 2-[3-cyano-4-(2-i-butoxy)phenyl]-4-methyl-5-thiazole-carboxylic acid, 135 ml of ethyl formiate were added. The mixture was heated under reflux until complete dissolution. The solution was cooled to 35° C., and the presence of a precipitate was observed during cooling. 65 ml of solvent were distilled under reduced pressure by maintaining the temperature. It was cooled at room temperature. It was kept at room temperature for 1 hour and then cooled to 0-5° C. This temperature was maintained for 1 hour. The product was filtered and dried for 15 hours at 60-65° C. 2.53 g of febuxostat as pure form II were obtained.

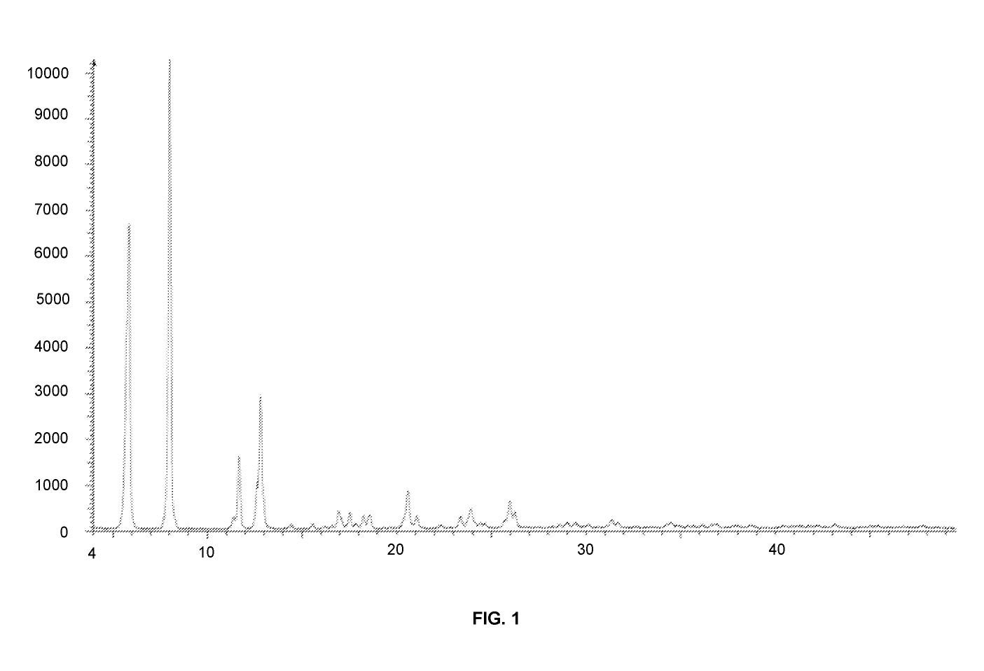

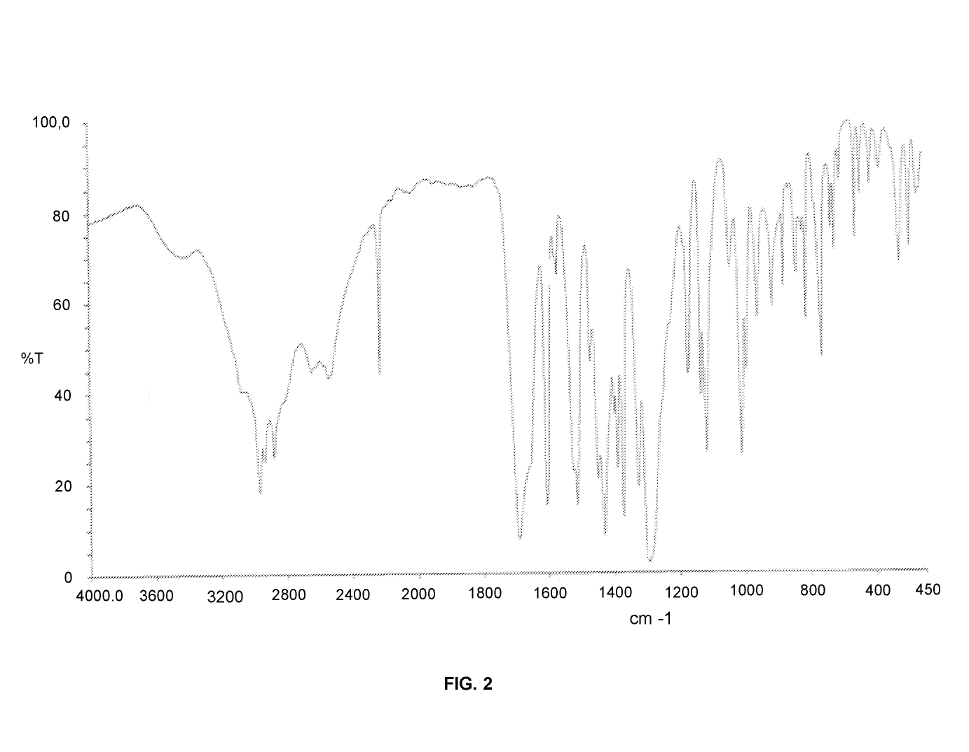

X-ray diagram (FIG. 1) and IR spectrum (FIG. 2) of any one of the samples prepared in Examples 1-3 were consistent with those reported in prior art.

1. A process for preparing the crystalline form II of febuxostat, comprising the following steps:
a) Dissolving febuxostat in a solvent selected from the group consisting of ethyl acetate, methyl acetate and ethyl formiate in a proportion from 10 to 60 ml of solvent per gram of solute, at a temperature between 50° C. and boiling temperature of the solution;
b) Forming the crystals by cooling the solution from step a) at a temperature between 20° C. and 45° C., optionally over a period of 0.5-2 hours under stirring;
c) Cooling the suspension from step b) at a temperature between 0° C. and 30° C. over a period of 0.5-3 hours; and
d) Isolating the crystalline form II of febuxostat by filtration and drying.

2. The process according to claim 1, step a), wherein the proportion of solvent per gram of solute is from 15 to 50 ml.

3. The process according to claim 1, step b), wherein the temperature ranges from 33° C. to 37° C.

4. The process according to claim 1, step b), wherein the period is 1 hour.

5. The process according to claim 1, which comprises -between step b) and step c)-increasing yield of step b) by eliminating 40-80% of the solvent by distillation under reduced pressure at a temperature between 30° and 40° C.

6. The process according to claim 5, wherein the temperature ranges from 33° C. to 37° C.
